(19) United States
(12) Patent Application Publication
(10) Pub. No.: US 2007/0038050 A1
(43) Pub. Date: Feb. 15, 2007

(54) Device for use with reflective pulse oximetry

(76) Inventor: Israel Sarussi, Yad Binyamin (IL)

Correspondence Address: DANIEL J SWIRSKY, 55 REUVEN ST., BEIT SHEMESH, 99544, IL

(21) Appl. No.: 11/502,623

(22) Filed: Aug. 11, 2006

Related U.S. Application Data
(60) Provisional application No. 60/707,556, filed on Aug. 12, 2005.

(51) Int. Cl. A61B 5/00

(52) U.S. Cl. 600/324

(57) ABSTRACT

A device for measuring and recording blood saturation and heart rate from a human digit, the device includes at least one light source, providing light directed toward the surface of the human digit, the light being absorbed by and reflected from within the human digit, a light detector spaced apart from the light source and being sensitive to intensity levels of the light reflected from within the human digit for producing intensity signals in accordance therewith, characterized by the device including an attaching component configured to be securely fastened to the human digit proximate the palm of the person whose blood saturation and heart rate is being measured. The attaching component is configured to exclude external light and to ensure that the performing component of the sensor presses into the tissue in such a way that the radiance source and detector are optically sealed off from each other.

### CROSS-REFERENCE TO RELATED APPLICATIONS

This application claims priority from U.S. Provisional Application No. 60/707.556, filed Aug. 12, 2005 and which is hereby incorporated by reference herein in its entirety.

### FIELD OF THE INVENTION

The present invention relates to a device for radiance based diagnostics using reflective pulse oximetry in general and, in particular, to a device for measuring and recording blood saturation and heart rate from a person's finger.

### BACKGROUND OF THE INVENTION

Radiance based diagnostics of body tissues involves radiating a body tissue and obtaining data relating to the transmittance or reflection of the radiated light from the tissue, for analysis of tissue constituents. Pulse oximetry is a method for measuring oxygen saturation in the blood, in which two or more wavelengths are radiated through an organ at a point where blood perfuses the organ. Reflective pulse oximetry employs at least one light source for radiating the organ and a least one detector for receiving the light reflected from the organ. The reflected light is analyzed for measuring the percent of saturated oxygen in the blood and heart rate.

Sleep disturbance is a well known problem affecting a major portion of the population. The inability to obtain a good nights sleep and the right quality of sleep pattern means that though people sleep, their sleep is not sufficiently restful and as a result, they wake up tired. Sleep interruption, which may also be caused by breathing problems are also common.

In order to diagnose these sleeping pattern disturbances, patients are sent to sleep laboratories that measure the sleeping pattern and analyze the different disturbance parameters.

One method for checking the patient's sleeping pattern includes the use of pulse oximeter devices that monitor and store the blood saturation level and the heart rate during the sleeping period. A partial analysis of the sleep quality and pattern may be achieved.

The pulse oximeter may be a standard off-the-shelf device or a watch shaped device; the latter device being attached to the patient's wrist. The sensor element may be positioned on the end of the patient's finger by a clip or an adhesive label. Generally, the sensor is connected to the main analytical component of the pulse oximeter device by a cable so that the sensor measurements may be forwarded for analysis.

Present day pulse oximeters have several disadvantages. Since the sensor is attached to a standard shelf pulse oximeter, the patient's movement is restricted. The reusable sensors are clumsy and limit the measurement quality, because they are liable to move and change their position on the finger whenever the patient moves while sleeping or become trapped in the bed sheets, for example. Present day devices suffer from a distortion of measurement commonly called motion artifact. Motion artifact is due to the differential motion between the sensor and the patient's finger as well as changes in pressure within the tissue.

Furthermore, the sensor on the patient's finger end limits the patient's actions. The sensor on the end of his finger restricts the use of that hand.

### SUMMARY OF THE INVENTION

It is an object of the present invention to provide a means of securely fastening a device for measuring and recording blood saturation and heart rate from a human digit.

There is thus provided a device for measuring and recording blood saturation and heart rate from a human digit. The device includes at least one light source, providing light directed toward the surface of the human digit, the light being absorbed by and reflected from within the human digit, a light detector spaced apart from the light source and being sensitive to intensity levels of the light reflected from within the human digit for producing intensity signals in accordance therewith, and a processing unit for processing the intensity signals received from the light detector for producing output signals.

The device is characterized by further including an attaching component configured to be securely fastened to the human digit proximate the palm of the person whose blood saturation and heart rate is being measured. The attaching component is configured to exclude external light and to ensure that the performing component of the sensor presses into the tissue in such a way that the radiance source and detector are optically sealed off from each other.

Furthermore, according an embodiment of the invention, the attaching component is fastened to the first phalanx of the human digit, the first phalanx being proximate the palm. Alternatively, the attaching component is fastened to the middle phalanx of the human digit.

Furthermore, according an embodiment of the invention, the attaching component includes a strip having a tab at one end for closing the strip around the digit by attaching the tab to body of the strip.

Furthermore, according an embodiment of the invention, the attaching component may include a strip having an adhesive side for affixing the light detector thereto, and a plurality of tabs for affixing the adhered to light detector to the underside of the end phalanx of the digit, said end phalanx being located at the fingernail end of the digit.

Furthermore, according an embodiment of the invention, the digital display may be in communication with the light detector. The digital display may be configured to display the results being measured by the light detector. The digital display may be attachable to the wrist of the person, whose blood saturation and heart rate is being measured.

### BRIEF DESCRIPTION OF THE DRAWINGS

The present invention will be understood and appreciated more fully from the following detailed description taken in conjunction with the appended drawings in which:

FIG. 1 is a view of display screen of the pulse oximeter attached to a patient's hand in accordance with an embodiment of the invention;

FIG. 2 is a view of the reflective sensor and pulse oximeter according to an embodiment of the present invention;

FIGS. 3A-3C are top, side and bottom elevational views of an embodiment of a strip configured to attach the sensor to a human digit; and

FIGS. 4A-4C are schematic views illustrating a method for fixing the adhesive component to the sensor and to the human digit.

### DESCRIPTION OF THE INVENTION

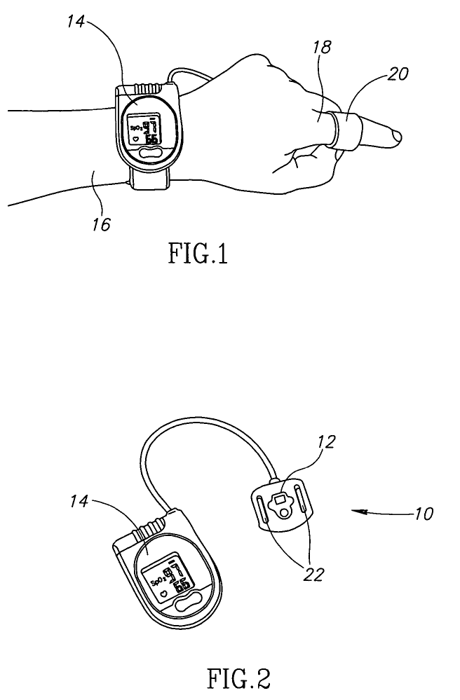

Reference is now made to FIGS. 1 and 2, which illustrate the pulse oximeter and reflective sensor, generally designated 10, attached to a patient's hand, in accordance with an embodiment of the invention.

The sensor is adapted to be in communication with a pulse oximeter (not shown) which is configured, in an exemplary embodiment, to be shaped like a watch 14 and adapted to fit a person's wrist 16 (as shown in FIG. 1).

According to an embodiment of the invention, the digital display may be in communication with the light detector and configured to display the results being measured by the light detector.

The pulse oximeter 12 may comprise electronic circuitry including a micro-processor which is in electronic communication with the performing component of the sensor for controlling the radiance source and the detector, and for performing analysis of the data received by the detector. The pulse oximeter and sensor may contain electronic circuitry similar to that described in U.S. Pat. No. 6,553,242 to the common assignees of the present application, which is incorporated herein by reference.

The electronic circuitry may be configured to record and store the measurements transmitted from the sensor, to perform data analysis and also to display the processed results such as the oxygen level and heat rate, for example. The electronic circuitry may also be configured to control the pressure of the sensor fastening so that an alert may be indicated, for example, should the pressure be too high or too low.

The present invention relates to a reflective pulse oximetry sensor which overcomes the disadvantages of prior art sensors. The sensor is configured to be located on the lower (closer to the palm) or medial knuckle of a patient's finger in order not to limit the patient's movements. The sensor may be strapped to any part of the circumference (internal, external or side face) of the finger by any suitable means, known in the art, such as a Velcro™ band or by plastic ring shaped device that may be spring loaded.

The sensor is configured for reflective oximetry and basically comprises a performing component includes at least one radiance source for radiating the tissue and at least one detector for detecting the rays reflected from the tissue.

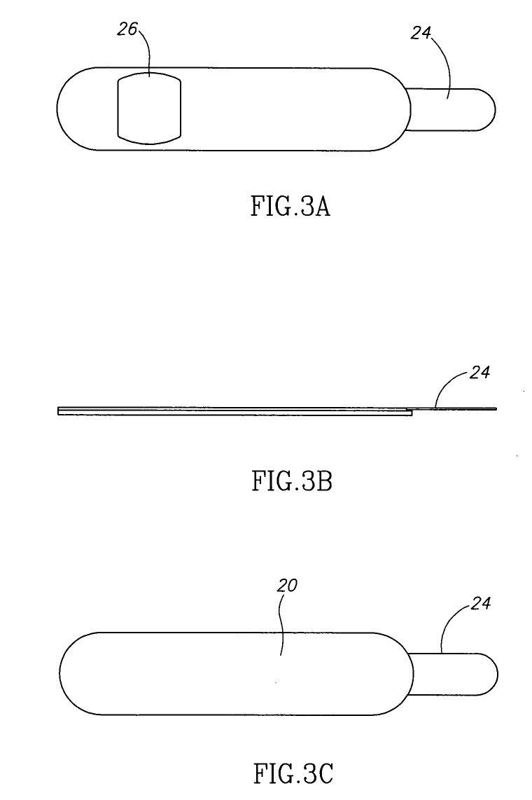

In one embodiment, the sensor may be fastened to the finger 18 (as shown in FIG. 1) by means of a Velcro™ type band 20 (best seen in FIG. 3), the sensor is enclosed within a housing having two slots 22 formed therein (FIG. 2). The Velcro™ band 20 is fed through the two slots 22 and using the Velcro type fastening the sensor unit is securely fastened in place around the finger 18.

The shape of the sensor is ergonomically adapted to fit around the finger. The use of the Velcro™ type band 20 enables the sensor to be placed in the preferred position for measurements to be taken and by suitably fastening the band, the sensor may be secured to the finger with the correct pressure.

The correct pressure maybe defined as the pressure which is high enough to ensure that any external light is blocked from the detector, in order to assure that only signals reflected from the tissue itself are registered. In other words, the sensor is configured to block any external light including light from the radiance source and light which maybe reflected from the skin itself. On the other hand, the pressure must not be too high so as to compress the small arteries (thereby nullifying the readings).

The inventors have realized that for reflective type oximetry measurements, it is essential to exclude external light from being received by the sensor. In addition, the sensor should only receive light reflected from within the tissue being measured. Consequently, the measurement of reflected light is best achieved by ensuring that the sensor is securely strapped to the finger close to the patient's palm, either on the first phalanx or second phalanx of the finger. In this way, the movement of the patient's finger is less likely to dislodge the sensor from its position and thus ensure higher reading accuracy.

Thus, the position of the sensor frees the nail end of the patient's finger and permits him to use his hand freely. The compactness of the sensor shape prevents the sensor from becoming trapped in the bed sheets and eliminates the problem of motion artifact.

The design of the sensor, as described above, ensures that, when the fastening component, such as the Velcro™ type band, is placed around the finger in contact with the tissue being measured, the performing component of the sensor presses into the tissue in such a way that the radiance source and detector are optically sealed off from each other.

According to an embodiment of the invention, a digital display may be connected to the processing unit of the sensor. The digital display may be configured in the shape of a watch, for example, or otherwise suitable attached to the wrist of the person whose blood saturation and heart rate is being measured.

Reference is now also made to FIGS. 3A-3C, which illustrate top, side and bottom elevational views of the strip 20 configured to attach the sensor/pulse oximeter to a person's finger 18. The strip 20 comprises a Velcro tab 24 at one end for closing the strip 20 around the figure by attaching the tab 24 to main body of the strip 20. The sensor 26 is suitable affixed to the underside of the strip 20.

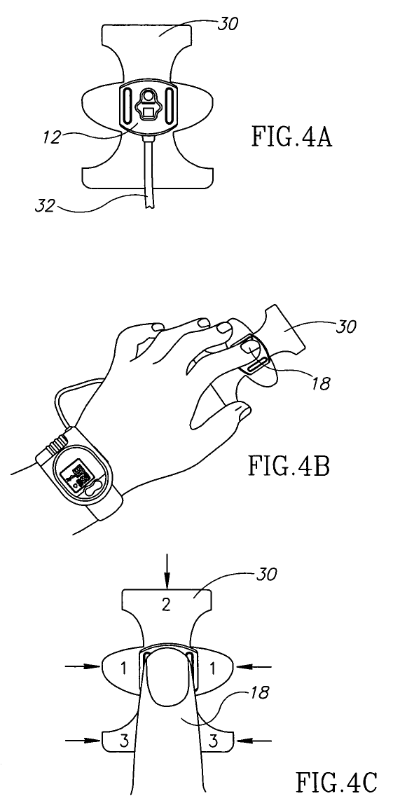

FIGS. 4A-4C illustrate an alternative exemplary adhesive component 30 for attaching the sensor/pulse oximeter to end of a person's finger 18. Adhesive component 30 is configured to securely adhere the sensor/pulse oximeter to the fingernail end of the digit and comprises any known in the art adhesive suitable for attaching to the human body. The shape of the adhesive component 30 is further configured to ensure that the performing component of the sensor presses into the tissue in such a way that the radiance source and detector are optically sealed off from each other, as described hereinabove. In contrast to the prior art, the configuration of adhesive component 30, ensures that motion artifact is substantially reduced and that the patient's actions are not limited.

The sensor 12 may be correctly attached to the digit 18 using the adhesive component 30, as follows:
a. place the adhesive component 30 on a clean dry surface with the adhesive side up;
b. place the sensor 12 in the center so that the cable 32 lies parallel with the digit 18;
c. position the index digit 18 and apply light pressure to the adhesive component 30;
d. place the tabs around the figures as ordered 1), 2) and 3).

It will be appreciated by persons skilled in the art that the present invention is not limited by what has been particularly shown and described herein above. Rather the scope of the invention is defined by the claims which follow:

1. A device for measuring and recording blood saturation and heart rate from a human digit, the device includes:
at least one light source, providing light directed toward the surface of the human digit, the light being absorbed by and reflected from within the human digit;
a light detector spaced apart from the at least one light source and being sensitive to intensity levels of the light reflected from within the human digit for producing intensity signals in accordance therewith; and
a processing unit for processing the intensity signals received from the light detector for producing output signals,
characterized by the device further includes:
an attaching component configured to be securely fastened to the human digit proximate the palm of the person whose blood saturation and heart rate is being measured, the attaching component configured to exclude external light and to ensure that the performing component of the sensor presses into the tissue in such a way that the radiance source and detector are optically sealed off from each other.

2. The device according to claim 1, wherein the attaching component is fastened to the first phalanx of the human digit, the first phalanx being proximate the palm.

3. The device according to claim 1, wherein the attaching component is fastened to the middle phalanx of the human digit.

4. The device according to claim 1, wherein the attaching component comprises a strip having a tab at one end for closing the strip around the digit by attaching the tab to body of the strip.

5. The device according to claim 1, wherein the attaching component comprises a strip having an adhesive side for affixing the light detector thereto, and a plurality of tabs for affixing the adhered to light detector to the underside of the end phalanx of the digit, said end phalanx being located at the fingernail end of the digit.

6. The device according to claim 1, further comprising a digital display in communication with the processing unit, said digital display configured to display the results being measured by the light detector.

7. The device according to claim 6, wherein said digital display is attachable to the wrist of the person, whose blood saturation and heart rate is being measured.
